(19) United States
(12) Patent Application Publication
(10) Pub. No.: US 2008/0226150 A1
(43) Pub. Date: Sep. 18, 2008

(54) X-ray CT imaging displaying method, X-ray CT image displaying apparatus, and X-ray CT apparatus

(75) Inventor: Tomoyuki Sadakane, Kyoto (JP)

Correspondence Address: Quinn Emanuel Urquhart Oliver & Hedges, LLP;Koda/Androlia, 10th Floor, 865 S. Figueroa Street, Los Angeles, CA, 90017, US

(73) Assignee: J. Morita Manufacturing Corporation

(21) Appl. No.: 12/072,363

(22) Filed: Feb. 26, 2008

(30) Foreign Application Priority Data
Feb. 27, 2007 (JP) 2007-47957
Feb. 22, 2008 (JP) 2008-41991

(51) Int. Cl. G06K 9/00; A61B 6/03

(52) U.S. Cl. 382/131; 378/19

(57) ABSTRACT

An X-ray CT image displaying method, an X-ray CT image displaying apparatus, and an X-ray CT apparatus are proposed and disclosed, in order to produce and display the occlusal plane surface image seen upwardly and downwardly from the inside of the oral cavity of the jaw so as to face each other on the basis of the occlusal surface, if the occlusal surface which is important for the dental diagnosis is specified on the basic image or the image data according to the X-ray absorption coefficient distribution information produced from X-ray projection image of the head of an object to be examined.

The X-ray CT image displaying method for displaying X-ray CT image of the head by X ray CT comprises: an occlusal surface specifying step of receiving an occlusal surface specifying in the X-ray image data obtained by X-ray CT of the head; an image producing step of producing from the X-ray image data an upward occlusal plane images seen upwardly from inside of the oral cavity of the jaw of the head and a downward occlusal plane image seen downwardly from the inside of the oral cavity both on the basis of the occlusal surface specified by the occlusal surface specifying step; and a displaying step of displaying the two upward and downward occlusal plane images produced by the image producing step in a manner that the upward and downward occlusal plane images face each other.

### TECHNICAL FIELD

The present invention relates to the improvement of an X-ray CT image displaying method, an X-ray CT image displaying apparatus, and an X-ray CT apparatus.

According to the method for example disclosed in JP-A-H08-215192 as a processing method of an X-ray CT image, the oblique sectional images which include the oblique line and is perpendicular to the panoramic curve is displayed by picturing the arbitrary oblique line in the layer image.

### BACKGROUND ART

According to the method disclosed in JP-A-H08-215192, if the layer image of the teeth of the object is produced and displayed from the continuous slice image obtained by an X-ray picturing apparatus and a plural of coordinated points to specify the panoramic curve is inputted on the layer image by operator, then the panoramic curve which connects the points are pictured on the layer image and the panoramic layer image which includes the pictured panoramic curve and is perpendicular to the layer image is produced and displayed from the continuous slice image mentioned above.

Also, according to the method disclosed in JP-A-H08-215192, the layer image and the panoramic layer image are pictured in parallel positions. On the panoramic layer image, the cursor for the operator to specify the arbitrary coordinated points are displayed so as to picturise the oblique line, and the coordinated points to picturise the oblique line are inputted by shifting the cursor.

When the coordinated points to picturise the oblique line are once inputted, then the oblique line which goes on the specified points and is perpendicular to the panoramic curve is picturised on the layer image, and the image of the oblique sectional image which is perpendicular to the panoramic curve and is along with the oblique line is produced and displayed by using said continuous slice image.

This kind of processing method is to display the layer image and the panoramic layer image in parallel positions, to picturise the oblique line on the layer image, and to display the oblique image which includes the oblique line and is perpendicular to the panoramic curve.

Also for example according to the method disclosed in JP-A-2006-500999, the sectional screen photograph of the upper jaw and the lower jaw are displayed in parallel positions, and the examples of the layout of the teeth-row-orthodontic and the wire which goes through the teeth-row-orthodontic are shown with comments on each screen photograph.

However, with these displaying methods, the operator has to understand the condition of the portion to be operated only from the layer plane image or the screen photograph, therefore it is difficult for the patient to understand the condition of the portion to be operated when the operator explains it to the patient.

Therefore, a displaying method or a displaying apparatus for dental use which is easy to see or to use not only for the operator but also for the patient is desired so that they can understand intuitively the condition of the affected area.

### SUMMARY OF THE INVENTION

The present invention has been proposed to solve the above-mentioned problems. A new X-ray CT image displaying method, a new X-ray CT image displaying apparatus, and a new X-ray CT apparatus, each of which can produce and displays the occlusal plane surface image seen upwardly and downwardly from the inside of the oral cavity of the jaw so as to face each other on the basis of the occlusal surface, if the occlusal surface which is important for the dental diagnosis is specified on the basic image or the image data according to the X-ray absorption coefficient distribution information produced from X-ray projection image of the head of an object to be examined.

In order to achieve the object of the present invention, according to the X-ray CT image displaying method of the claim 1, the X-ray CT image displaying method for displaying X-ray CT image of the head of an object by X-ray CT comprises: an occlusal surface specifying step of receiving an occlusal surface specifying in the X-ray image data obtained by X-ray CT of the head of an object to be examined; an image producing step of producing from the X-ray image data an upward occlusal plane image seen upwardly from inside of the oral cavity of the jaw of the head of an object to be examined and a downward occlusal plane image seen downwardly from the inside of the oral cavity both on the basis of the occlusal surface specified by the occlusal surface specifying step; and a displaying step of displaying the two upward and downward occlusal plane images produced by the image producing step in a manner that the upward and downward occlusal plane images face each other.

According to this X-ray CT image displaying method of the claim 1, if the occlusal surface is specified in the X-ray image data obtained by X-ray CT of the head of an object, then the upward occlusal plane image seen upwardly from inside of the oral cavity of the jaw and a downward occlusal plane image seen downwardly from the inside of the oral cavity are produced from the X-ray image data and these upper and lower occlusal plane surface images are displayed so as to face each other.

In this X-ray CT image displaying method, the upper and lower occlusal plane surface images are displayed in the manner that the jaw is opened like a spread hard fish upwardly and downwardly both on the basis of the occlusal surface. The occlusal plane surface image displayed according to the present invention only has to be an image on the basis of the X-ray absorption coefficient distribution information obtained by the X-ray CT and the image reconstruction. Then this X-ray CT image displaying method can display at least one of the X-ray layer image and the volume rendering image, or both.

According to the X-ray CT image displaying method of the claim 2 in the X-ray CT image displaying method as set forth in claim 1, in the occlusal surface specifying step, at least the surface specifying of a first occlusal surface and a second occlusal surface both of which are different from each other are received, and in the image producing step, the upward and downward occlusal plane images are produced from the X-ray image data on the basis of at least the first occlusal surface and the second occlusal surface.

According to this X-ray CT image displaying method of the claim 2, the upward and downward occlusal plane images are produced from the X-ray image data on the basis of the first occlusal surface and the second occlusal surface respectively both of which are different from each other. Therefore, because the operator can observe the detail construction of the specified layer plane by specifying the each of the first and second occlusal surfaces as for the layer plane of the jaw of the object which he wants to observe, then the projecting shape of the root portion of the tooth, the layer plane of the tooth, the condition of the filling, the depth of the decayed tooth at the upper jaw and the lower jaw of the object, can be understood more intuitively and conveniently. Alternatively, the first and second occlusal surfaces may be specified by the operator at will, or may be specified according to the condition prepared in advance. For example, first and second occlusal surfaces may be specified as the occlusal surfaces which are separated at a specified distance on the basis of one certain occlusal surface. Further, the first and second occlusal surfaces may be shifted in conjunction with each other in the manner that one of the first and second occlusal surfaces is shifted, when another is shifted, or contrarily, the first and second occlusal surfaces may be specified independently for example in the manner that one of them is not shifted, even when another is shifted.

According to the X-ray CT image displaying method of the claim 3 in the X-ray CT image displaying method as set forth in claim 1 or 2, the occlusal surface specifying step comprises: a step of producing and displaying at least one of the front sectional image, the side sectional image, and the basic volume rendering image as a basic image from the X-ray absorption coefficient distribution information obtained from the X-ray CT of the head; and a step of receiving an operation of a cursor to specify the occlusal surface on at least one of the front sectional image, the side sectional image, and the basic volume rendering image.

According to the X-ray CT image displaying method of the claim 3, the occlusal surface is specified by the cursor operation on the at least one image of the front sectional image, the side sectional image, and the basic volume rendering image. The front sectional image and the side sectional image are the sectional images produced from the X-ray absorption coefficient distribution information while the flat plane parallel to the body axis of the object is set as the layer plane. In this specification hereinafter, the sectional image which is produced as the layer plane which is a perpendicular plane to the cross direction of the object is mentioned as the “front sectional image” and the sectional image which is produced as the layer plane which is a parallel plane to the cross direction of the object is mentioned as the “side sectional image”.

According to the X-ray CT image displaying method of the claim 4 in the X-ray CT image displaying method as set forth in claim 1 or 2. The X-ray image data is an X-ray image data obtained by picturing the object which bites a bite-block. The X-ray CT image displaying method further comprises a step of reconstructing the X-ray image data for producing an X-ray absorption coefficient distribution information of the head of an object to be examined as a preprocessing for receiving the occlusal surface specifying. And in the occlusal surface specifying step, the bite-block is extracted from the X-ray absorption coefficient distribution information by image-analysis to specify the occlusal surface, and figures out the layer image corresponding to the specified occlusal surface from the X-ray absorption coefficient distribution information thus obtained.

According to this X-ray CT image displaying method of the claim 4, the occlusal surface is specified by extracting the bite block from the X-ray absorption coefficient distribution information of the head by the image-analysis and the specified occlusal surface corresponding to the layer plane is figured out from the X-ray absorption coefficient distribution information.

According to the X-ray CT image displaying method of the claim 5 in the X-ray CT image displaying method as set forth in claim 1 or 2, the X-ray CT image displaying method further comprises a step of reconstructing an X-ray projection image of the head to produce an X-ray absorption coefficient distribution information as a preprocessing for receiving the occlusal surface specifying. And in the occlusal surface specifying step, the occlusal surface is specified by image-analysis, and the layer image corresponding to the specified occlusal surface thus specified is figured out from the X-ray absorption coefficient distribution information.

According to this X-ray CT image displaying method of the claim 5, the occlusal surface is specified from the X-ray absorption coefficient distribution information by the image-analysis and the layer plane corresponding to the specified occlusal surface is figured out from the X-ray absorption coefficient distribution information.

According to the X-ray CT image displaying method of the claim 6 in the X-ray CT image displaying method as set forth in claim 1 or 2, the X-ray CT image displaying method further comprises the color-code displaying step of displaying the upward and downward occlusal plane images by the color-coding according to the X-ray absorption coefficient after producing the upward and downward occlusal plane images.

Each X-ray CT image displaying apparatus of the claim 7-claim 12 are to realize the three dimensional displaying method for tooth jaw layer plane of the claim 1-claim 6.

According to the X-ray CT image displaying apparatus of the claim 7, the X-ray CT image displaying apparatus for displaying X-ray CT image of the head by X ray CT comprises: an occlusal surface specifying means for specifying an occlusal surface in the X-ray image data obtained by X-ray CT for the head; an image producing means for producing from the X-ray image data an upward occlusal plane image seen upwardly from inside of the oral cavity of the jaw and a downward occlusal plane image seen downwardly from the inside of the oral cavity both on the basis of the occlusal surface specified by the occlusal surface specifying means; and a displaying means for displaying the upward and downward occlusal plane images produced by the image producing means in a manner that the upward and downward occlusal plane image face each other.

The occlusal plane images specified by the apparatus of the claim 7 only has to be the image on the basis of the X-ray absorption coefficient distribution information obtained by reconstructing and by the X-ray CT and this X-ray CT image displaying method may display at least one of the X-ray layer image and the volume rendering image, or both.

According to the X-ray CT image displaying apparatus of the claim 8 in the X-ray CT image displaying apparatus as set forth in claim 7, the occlusal surface specifying means specifies at least a first occlusal surface and a second occlusal surface, both of which are different from each other, and the image producing means produces the upward and downward occlusal plane images from the X-ray image data on the basis of at least the first occlusal surface and the second occlusal surface.

According to the X-ray CT image displaying apparatus of the claim 9 in the X-ray CT image displaying apparatus as set forth in claim 7 or 8, the occlusal surface specifying means displays at least one of the front sectional image, the side sectional image, and the basic volume rendering image of the head as a basic image to specify said occlusal surface, each of the images being produced from the X-ray absorption coefficient distribution information produced by the X-ray CT of the head, and specifies the occlusal surface by the operation of a cursor on at least one of the front sectional image, the side sectional image, and the basic volume rendering image.

According to the X-ray CT image displaying apparatus of the claim 10 in the X-ray CT image displaying apparatus as set forth in claim 7 or 8, the X-ray image data is an X-ray image data obtained by picturing the object which bites a bite-block, the X-ray CT image displaying apparatus further comprises an X-ray absorption coefficient distribution information producing means for reconstructing the X-ray image data to produce an X-ray absorption coefficient distribution information of the head, and said occlusal surface specifying means extracts the bite-block from the X-ray absorption coefficient distribution information by image-analysis to specify the occlusal surface, and figures out the layer image corresponding to the specified occlusal surface from the X-ray absorption coefficient distribution information thus obtained.

According to this X-ray CT image displaying apparatus of the claim 10, the occlusal surface specifying means extracts the bite-block from the X-ray absorption coefficient distribution information by image-analysis to specify the occlusal surface, and figures out the layer image corresponding to the specified occlusal surface from the X-ray absorption coefficient distribution information thus obtained.

According to the X-ray CT image displaying apparatus of the claim 11 in the X-ray CT image displaying apparatus as set forth in claim 7 or 8: the X-ray CT image displaying apparatus further comprises an X-ray absorption coefficient distribution information producing means for reconstructing an X-ray projection image of the head to produce an X-ray absorption coefficient distribution information, and the occlusal surface specifying means specifies the occlusal surface by image-analysis to specify the occlusal surface, and figures out the layer image corresponding to the specified occlusal surface from the X-ray absorption coefficient distribution information.

According to this X-ray CT image displaying apparatus of the claim 11, in the occlusal surface specifying means, the image-processing means specifies the occlusal surface of the object by image-analysis from the X-ray absorption coefficient distribution information, and the occlusal plane surface images upwardly and downwardly seen from the inside of the oral cavity of the jaw on the basis of the specified occlusal surface is produced and displayed so as to face each other.

According to the X-ray CT image displaying apparatus of the claim 12 in the X-ray CT image displaying apparatus as set forth in claim 7 or 8, the displaying means displays the upward and downward occlusal plane images by the color-coding according to the X-ray absorption coefficient after producing the upward and downward occlusal plane images by the image producing means.

Further, According to the X-ray CT apparatus of the claim 13, the X-ray CT apparatus comprises: an X-ray CT main unit at least having an X-ray detecting portion and an X-ray producing portion which produces an X-ray cone beam; the X-ray CT image displaying apparatus as set forth in claims 7 or 8; and the X-ray CT main unit sets the projection area of the head of an object to be examined between the X-ray producing portion and the X-ray detecting portion, and rotates both of the X-ray producing portion and the X-ray detecting portion around the projection area for obtaining the X-ray CT image of a head of the object.

According to the claim 1, the occlusal surface specifying step receives an occlusal surface specifying in the X-ray image data obtained by X-ray CT of the head, the image producing step produces from the X-ray image data the upward and downward occlusal plane image on the basis of the occlusal surface specified by the occlusal surface specifying step and the displaying step displays the two upward and downward occlusal plane images so as to face each other. Therefore, the layer plane of the occlusal surface which is preferable to see and important as the dental diagnosis and where the upper and lower teeth are in mesh, can be specified at will. Further the two upward and downward occlusal plane images seen upwardly and downwardly from the inside of the oral cavity both on the basis of the specified occlusal surface can be produced and displayed so as to face each other.

Thus, the image of the upper jaw and the lower jaw are observed at once in the up and down positions or right and left positions by displaying the upper and lower occlusal plane surface images so as to face each other. The condition that the image of the upper jaw and the lower jaw are observed at once in the parallel positions is very close to the condition that the operator observes the inside of the oral cavity of the patient who actually opens his mouth. Therefore, both of the patient and the operator can understand the inside condition of the oral cavity or the position of the teeth intuitively while watching the X-ray image data of the object and confirming the upper and lower occlusal plane surface images of the jaw on the basis of the occlusal surface where is preferred to be seen,

According to the claim 2, in the image producing step, the upward and downward occlusal plane images are produced on the basis of at least the first occlusal surface and the second occlusal surface both of which are different from each other. Therefore, the operator can understand the inside condition of the oral cavity and the position of the teeth more precisely and intuitively. For example the projecting shape of the root portion of the tooth, the layer plane of the tooth, the condition of the filling, the depth of the decayed tooth at the upper jaw and the lower jaw of the object, can be understood more intuitively and conveniently. If the first and second occlusal surfaces may be specified by the operator at will, then the preferable position in the root portion of the tooth for example can be observed. Alternatively, according to the condition prepared in advance, for example, if the occlusal surfaces which are separated at a specified distance on the basis of one certain occlusal surface is constituted as the first and second occlusal surfaces, then the occlusal surface can be specified conveniently without troubles. Further, if the first and second occlusal surfaces are shifted in conjunction with in the manner that one of the first and second occlusal surfaces is shifted, when another is shifted, then for example the root portion of the tooth portion of the upper jaw and the lower jaw at the same depth can be observed at the same time. Or if each of the first and second occlusal surfaces is specified independently in the manner that one of the first and second occlusal surfaces is not shifted, even when another is shifted, then the most preferable portions to see respectively of the upper jaw and the lower jaw can be selected and displayed at the same time, so that the operator can usefully explain to the patient.

According to the claim 3, the occlusal surface specifying step comprises a step of producing and displaying at least one of the front sectional image, the side sectional image, and the basic volume rendering image as a basic image from the X-ray absorption coefficient distribution information obtained from the X-ray CT of the head, and a step of receiving an operation of a cursor to specify the occlusal surface on at least one of the front sectional image, the side sectional image, and the basic volume rendering image. Therefore, the operator can specify the occlusal surface by the operation of a cursor on at least one of the front sectional image, the side sectional image, and the basic volume rendering image. It is useful for the operator and the patient to be able to display the preferable occlusal plane surface image precisely with a simple operation like a cursor operation.

According to the claim 4, by using the bite block which is bitten by the object, the X-ray absorption coefficient distribution information is extracted by image-analysis so as to specify the occlusal surface, and the layer image corresponding to the specified occlusal surface is figured out from the X-ray absorption coefficient distribution information. Therefore, the occlusal surface of the object is automatically specified and the occlusal plane surface image on the basis of the layer plane corresponding to the specified occlusal surface is produced and displayed so as to face each other, so that it is convenient for the operator

According to the claim 5, the occlusal surface is specified by the image-analysis of the X-ray absorption coefficient distribution information of the object, the layer plane corresponding to the specified occlusal surface is figured out without the bite-block or the operation to specify the occlusal surface by the cursor. Therefore the upper and lower occlusal plane surface images seen from the oral cavity of the jaw on the basis of the layer plane is produced and displayed more conveniently so as to face each other.

According to the claim 6, the occlusal plane images is displayed by the color-coding according to the X-ray absorption coefficient after the upward and downward occlusal plane images of the inside of the oral cavity of the jaw of the object are produced. Therefore, the conditions inside of the oral cavity, such as the teeth, the dental pulp, the gingiva, the implant, and the filling can be understood more intuitively and conveniently.

According to the claim 7, the X-ray CT image displaying apparatus comprises: an occlusal surface specifying means for specifying an occlusal surface in the X-ray image data obtained by X-ray CT for the head; an image producing means for producing from the X-ray image data an upward occlusal plane image seen upwardly from inside of the oral cavity of the jaw and a downward occlusal plane image seen downwardly from the inside of the oral cavity both on the basis of the occlusal surface specified by the occlusal surface specifying means; and a displaying means for displaying the upward and downward occlusal plane images produced by the image producing means in a manner that the upward and downward occlusal plane image face each other. Therefore, the X-ray CT image displaying method of the claim 1 can be realized.

According to the claim 8, the image producing means produces the upward occlusal plane image seen upwardly from inside of the oral cavity of the jaw of the head of an object and the a downward occlusal plane image seen downwardly from the inside of the oral cavity from the X-ray image data on the basis of at least the first occlusal surface and the second occlusal surface. Therefore, the X-ray CT image displaying method of the claim 2 can be realized.

According to the claim 9, the occlusal surface specifying means displays at least one of the front sectional image, the side sectional image, and the basic volume rendering image of the head as a basic image to specify the occlusal surface, each of the images being produced from the X-ray absorption coefficient distribution information produced by the X-ray CT of the head, and specifies the occlusal surface by the operation of a cursor on at least one of the front sectional image, the side sectional image, and the basic volume rendering image. Therefore, the X-ray CT image displaying method of the claim 3 can be realized.

According to the claim 10, the X-ray CT image displaying apparatus further comprises an X-ray absorption coefficient distribution information producing means for reconstructing the X-ray image data to produce an X-ray absorption coefficient distribution information of the head, and the occlusal surface specifying means extracts the bite-block from the X-ray absorption coefficient distribution information by image-analysis to specify the occlusal surface, and figures out the layer image corresponding to the specified occlusal surface from the X-ray absorption coefficient distribution information thus obtained. Therefore, the X-ray CT image displaying method of the claim 4 can be realized.

According to the claim 11, the X-ray CT image displaying apparatus further comprises an X-ray absorption coefficient distribution information producing means for reconstructing an X-ray projection image of the head to produce an X-ray absorption coefficient distribution information, and the occlusal surface specifying means specifies the occlusal surface by image-analysis to specify the occlusal surface, and figures out the layer image corresponding to the specified occlusal surface from the X-ray absorption coefficient distribution information. Therefore, the X-ray CT image displaying method of the claim 5 can be realized.

According to the claim 12, the displaying means displays the upward and downward occlusal plane images by the color-coding according to the X-ray absorption coefficient after producing the upward and downward occlusal plane images by the image producing means. Therefore, the X-ray CT image displaying method of the claim 4 can be realized.

According to the claim 13, the X-ray CT apparatus comprises at least one of the X-ray CT image displaying apparatuses as set forth in claims 7 or 8. Therefore, the X-ray CT apparatus that has the effect of claims 7-12 can be proposed.

### BRIEF DESCRIPTION OF THE DRAWINGS

FIG. 1 shows a displaying example of the present invention, that shows an example of the displaying embodiment of the basic image and the occlusal plane surface image upwardly and downwardly seen from the inside of the oral cavity of the jaw in the displaying portion.

FIG. 2 shows the flow chart explaining an example of the displaying method of the X-ray CT image of the present invention.

FIG. 3 shows a drawing explaining the basic principle to produce the front sectional image and the side sectional image as the basic image from the X-ray image data of the head.

FIG. 4 shows a drawing explaining the basic principle to produce the volume rendering image upwardly and downwardly seen from the inside of the oral cavity of the jaw from the X-ray absorption coefficient distribution information of the head.

FIG. 5 shows the other example of the occlusal surface specifying step to be explained.

FIG. 6 shows the other example of the embodiment, in which the occlusal plane surface images are displayed so as to face each other.

FIG. 7 shows the other example of the embodiment displaying the basic image and the occlusal plane surface images upwardly and downwardly seen from the inside of the oral cavity of the jaw in the displaying portion.

FIG. 8 shows the flow chart to explain X-ray CT image displaying method which specifies the occlusal surface automatically as the other example of the X-ray CT image displaying method of the present invention.

FIG. 9 shows the other example of the displaying embodiment of the basic image and the occlusal plane surface images upwardly and downwardly seen from the inside of the oral cavity of the jaw on the displaying portion.

FIG. 10 shows the flow chart to explain the other example of the X-ray CT image displaying method of the present invention, in which the occlusal surface is automatically specified.

FIG. 11 shows the schematic drawing of the X-ray absorption coefficient distribution information of the side head of the object to explain the automatic specifying of the occlusal surface.

FIG. 12 shows the block diagram to explain the basic construction of the X-ray CT apparatus M including the X-ray CT image displaying apparatus of the present invention.

### BEST MODE FOR CARRYING OUT THE INVENTION

The embodiments of the present invention are explained as follows.

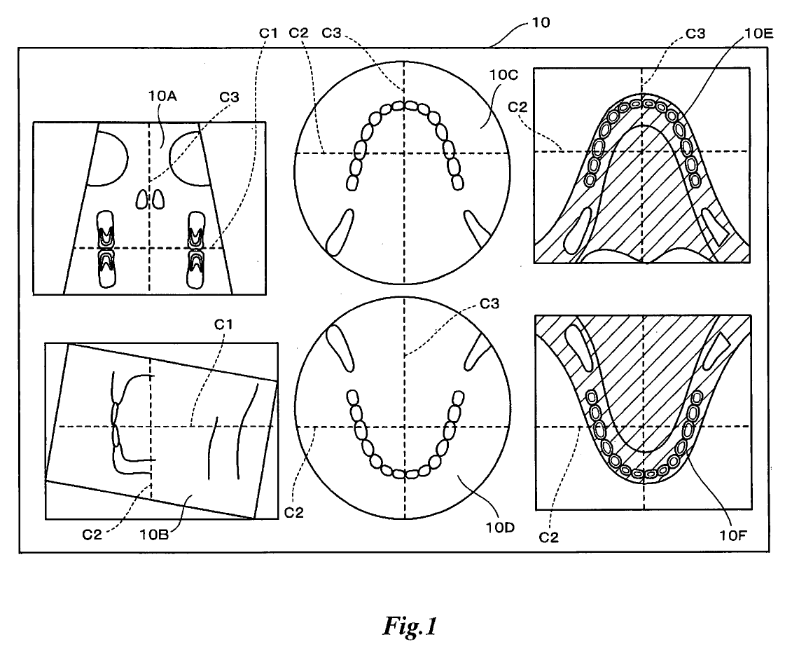

FIG. 1 shows an example of the displaying embodiment of the basic image and the occlusal plane surface images upwardly and downwardly seen from the inside of the oral cavity of the jaw in the displaying portion. The displaying portion 10 mentioned here can carry out the displaying method of the present invention, if the displaying portion 10 is provided with a general-purpose displaying apparatus such as a liquid crystal display.

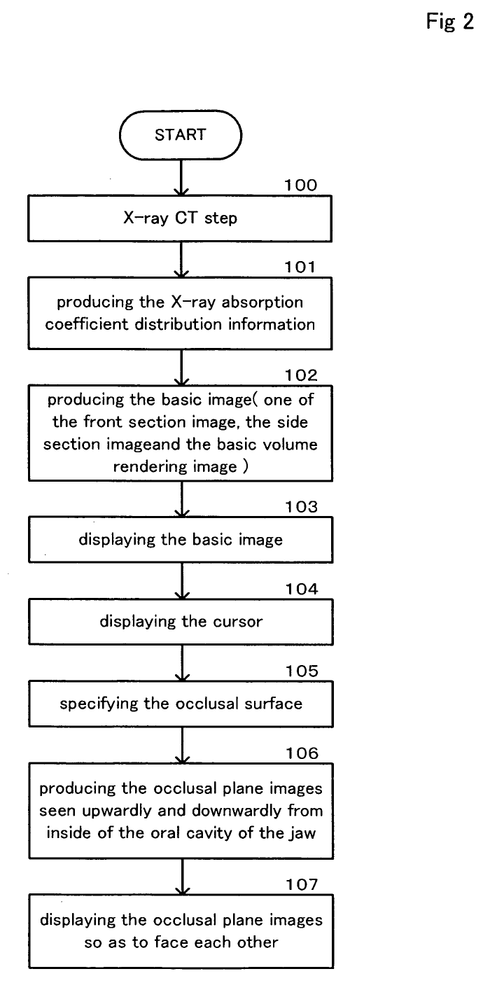

FIG. 2 shows the flow chart (100-107) explaining an example of the displaying method of the X-ray CT image of the present invention.

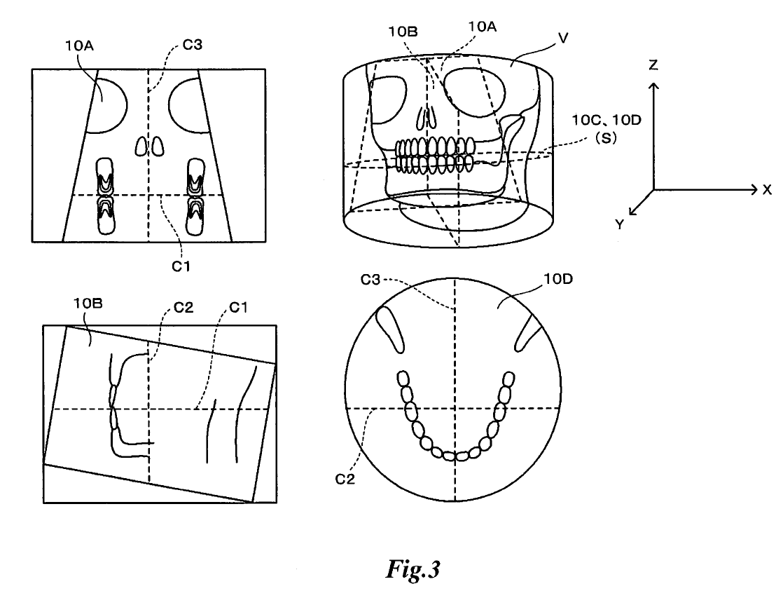

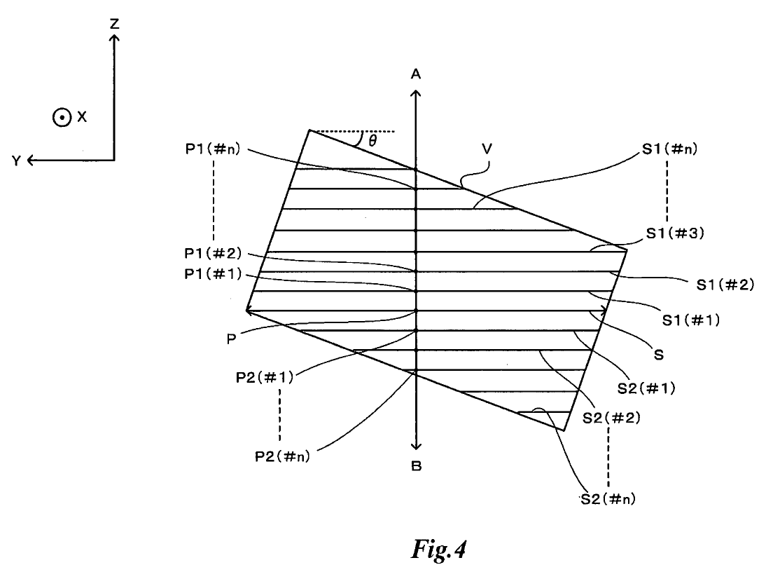

FIG. 3 shows a drawing explaining the basic principle to produce the basic image from the X-ray image data of the object. FIG. 4 shows a drawing explaining the basic principle to produce the volume rendering image upwardly and downwardly seen from the inside of the oral cavity of the jaw from the X-ray image data of the head.

The X-ray CT image displaying method of the present invention comprises: an occlusal surface specifying step of receiving an occlusal surface specifying in the X-ray image data obtained by X-ray CT of the head of an object to be examined; an image producing step of producing from the X-ray image data an upward occlusal plane image seen upwardly from inside of the oral cavity of the jaw of the head of an object to be examined and a downward occlusal plane image seen downwardly from the inside of the oral cavity both on the basis of the occlusal surface specified by the occlusal surface specifying step; and a displaying step of displaying (displaying so as to face each other) the two upward and downward occlusal plane images produced by the image producing step in a manner that the upward and downward occlusal plane images face each other.

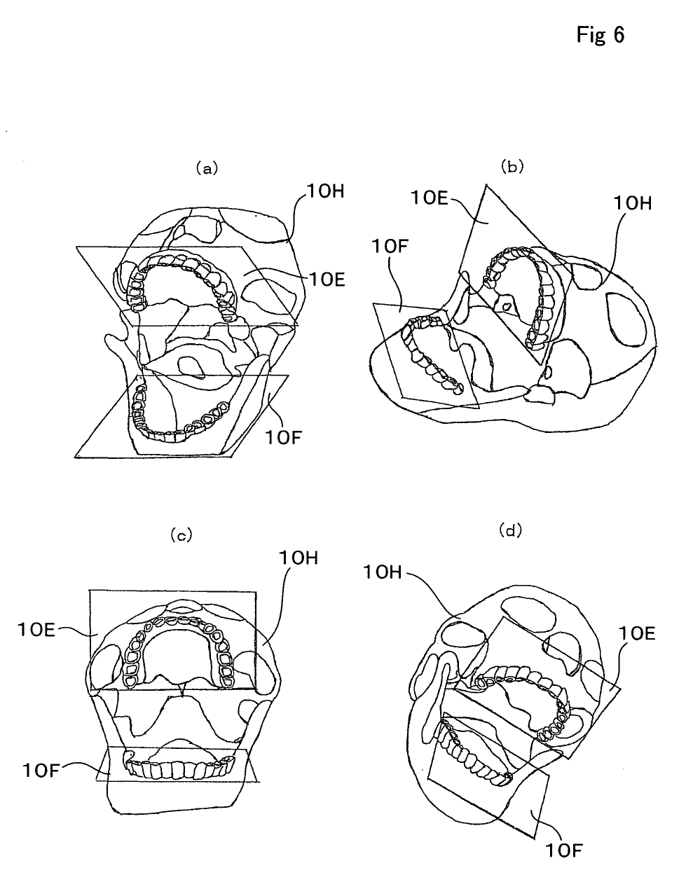

The upper and lower occlusal plane surface images includes horizontal sectional image upwardly and downwardly seen from the inside of the oral cavity of the jaw 10C, 10D (FIG. 1) in addition to the volume rendering image 10E, 10F (FIG. 1 FIG. 6) obtained by the volume rendering upwardly and downwardly from the jaw on the basis of the occlusal surface S respectively.

Also, the embodiment to display so as to face each other includes the embodiment to display the volume rendering image 10E, 10F in which the jaw is opened by less than 180 degree as shown in FIG. 6, in addition to the embodiment to display the volume rendering image upwardly seen from the inside of the oral cavity of the jaw 10E (or the horizontal sectional image 10C) and the volume rendering image downwardly seen from the inside of the oral cavity of the jaw 10F (or horizontal sectional image 10D) in which the jaw is opened by less than 180 degree, both on the basis of the occlusal surface S as shown in FIG. 1.

In the case of the embodiment to display the images so as to face each other in which the jaw is opened by less than 180 degree, the volume rendering image 10E and 10F are displayed in parallel position, so that the lower jaw is opened and closed against the upper jaw around the temporomandibular joint so as to combine the volume rendering images 10E, 10F as a volume rendering image which becomes a model of the head of the object (FIG. 6).

In this case the volume rendering image 10E, 10F may be constituted to be able to be adjusted by shifting, so that the operator can slightly adjust the displayed position of the volume rendering image 10E, 10F at will while watching the position of the upper and lower temporomandibular joint.

According to present invention, the effect that the operator or the patient can see and understand easily and intuitively the provided image which displays the desired upper and lower occlusal plane surface images so as to face each other is achieved, even when the jaw opens at any angle such as 180 degree or less than 180 degree.

The upper and lower occlusal plane surface images may be displayed in upper and lower position in parallel as shown in FIG. 1., however the present invention is not limited to this embodiment. The occlusal image may be displayed in right and left positions. For example, the front teeth and the cheek teeth of the upper jaw may be displayed respectively in left and right positions, and the front teeth and the cheek teeth of the lower jaw may be displayed respectively in right and left positions.

As shown in FIG. 1, the displaying portion 10 shows the front sectional image 10A and the side sectional image 10B as the basic images which is produced from the X-ray absorption coefficient distribution information V obtained by backward projection and by reconstructing the X-ray image data (X-ray projection image) of the head of the object, where the X-ray image data being obtained by X-ray CT.

FIG. 1 shows the eyepit, the nasal cavity, the left and right first molar tooth and the alveolar bone of the first molar tooth respectively of the object as a front sectional image 10A. Also FIG. 1 shows the front teeth of the object and their alveolar bone as a side sectional image 10B.

The side sectional image 10B shows and emphasizes the front teeth and the alveolar bone of the object in order to explain the method in which the operator specifies the occlusal surface S of the object on the side sectional image 10B. However, the eyepit or the nasal cavity of the object may be further displayed to specify the occlusal surface S of the object. According to the present embodiment, the image of the front teeth and the alveolar bone are not shown, because they are not essential in the present invention in which the occlusal surface S of the object is specified on the basic image to display the upward and downward plane occlusal images so as to face each other on the basis of the specified occlusal surface S.

Further, the basic volume rendering image 10G mentioned hereinafter may be produced and displayed as a basic image.

Further, the displaying portion 10 displays the horizontal sectional image 10C and the horizontal sectional image 10D so as to face each other, both of which are produced on the basis of the occlusal surface S specified by the operator, where the horizontal sectional image 10C is seen upwardly from the inside of the oral cavity of the jaw and the horizontal sectional image 10D is seen downwardly from the inside of the oral cavity.

Further, displaying portion 10 displays the volume rendering image 10E seen upwardly from the inside of the oral cavity of the jaw and the volume rendering image 10F seen downwardly from the inside of the oral cavity, both volume rendering image being produced on the basis of the occlusal surface S specified by the operator.

The horizontal sectional image 10C, 10D and the volume rendering image 10E, 10F, both of which constitute the occlusal plane surface images of the present invention, are not necessarily displayed at the same time. Either of them may be sufficient. When either of them is displayed, either the horizontal sectional image 10C, 10D, or the volume rendering image 10E, 10F may be preferentially displayed, or alternately, the operator or the patient may arbitrarily select the images to be displayed. Further, the images may be sequentially displayed, for example in the manner that the only horizontal sectional image 10C (or 10D), or the volume rendering image 10E (or 10F) is displayed at first soon after the X-ray CT, then the occlusal surface S is specified as mentioned hereinafter, and then the rest of the horizontal sectional image 10D (or 10C), or the volume rendering image 10F (or 10E) is displayed.

In the X-ray absorption coefficient distribution information V mentioned hereinafter, the occlusal surface S of the object inclines against the horizontal direction by 0 degrees (approximately 4.5-6.5 degrees) forwardly seen from the object, therefore the X-ray absorption coefficient distribution information V is converted to be inclined backwardly seen from the object by the image-processing in order to make the occlusal surface S of the object horizontal.

With this image-processing, the side sectional image 10B is displayed so as to be inclined by 0 degrees backwardly seen from the object. This displaying embodiment is only an example, therefore, if the occlusal surface S of the object is approximately horizontal at the time of the X-ray CT, then the further image-processing to incline the image by θ degree is not necessary. Alternately, even if the occlusal surface S of the object is inclined for example by θ degrees against the horizontal direction, the horizontal section setting cursor C1 can be displayed so as to incline by θ degrees at the time when the occlusal surface S is specified by the cursor as mentioned hereinafter.

The front sectional image 10A and the side sectional image 10B comprise at least a horizontal section setting cursor C1 to specify the horizontal section, the longitudinal section setting cursor C2 to specify the longitudinal section, and the cross section setting cursor C3 to specify the cross section, in order to specify the layer plane of the X-ray absorption coefficient distribution information V.

In the present embodiment, all of these three cursors are explained as an example, although all of them are not necessarily provided at the same time. The horizontal section setting cursor C1 has the function to specify the occlusal surface S in the occlusal surface specifying step as mentioned hereinafter.

The side sectional image 10B shows the horizontal section setting cursor C1 which is shifted in an up and down directions on the side sectional image 10B and the longitudinal section setting cursor C2 which is shifted in a transverse direction on the side sectional image 10B. The horizontal section is specified by shifting the horizontal section setting cursor C1 and the longitudinal section is specified by shifting the longitudinal section setting cursor C2.

In the occlusal surface specifying step, the occlusal surface S where the teeth of the object is bite is specified as a horizontal section by shifting the horizontal section setting cursor C1 as mentioned hereinafter.

When the horizontal section setting cursor C1 is shifted by operation to specify the occlusal surface S in the occlusal surface specifying step, the horizontal sectional image upwardly seen from the inside of the oral cavity of the jaw 10C and the horizontal sectional image downwardly seen from the inside of the oral cavity of the jaw 10D are produced and displayed so as to face each other on the basis of the specified occlusal surface S.

The horizontal sectional image 10C is the upper jaw's horizontal sectional image sliced by the horizontal section specified by the horizontal section setting cursor C1, and the horizontal sectional image 10D is the lower jaw's horizontal sectional image sliced by the horizontal section specified by the horizontal section setting cursor C1. Because only one horizontal section is specified, the horizontal sectional image 10C, 10D are in the so called miller image relation in the figure where the same layer plane image is shown in up-and-down positions.

If the longitudinal section setting cursor C2 is shifted by the operation to specify the longitudinal section on the side sectional image 10B, then the front sectional image 10A is displayed selectively according to the specified longitudinal section.

Further, if the horizontal section setting cursor C1 and the cross section setting cursor C3 are shifted by the operation to specify the horizontal section and the cross section respectively on the front sectional image 10A, then the side sectional image 10B and the horizontal sectional image 10C, 10D are displayed selectively according to the specified horizontal section and the specified cross section respectively.

In the occlusal surface specifying step, not only on the side sectional image 10B, but also on the front sectional image 10A, the occlusal surface S can be specified by shifting the horizontal section setting cursor C1 as an operation. It is not necessary to say that each cursor C1, C2, C3 are not limited to the dotted line as shown in FIG. 1 but they can be a full line or an arrow, and so on.

An example of the X-ray CT image displaying method of the present invention shown in the flow chart of FIG. 2 is as follows. The X-ray absorption coefficient distribution information V of the head is produced by backward projection and by reconstructing the X-ray image data of the head of an object with the conventional method, while the X-ray image data is obtained by the X-ray CT step 100. Then the occlusal surface specifying step 101-105, the image producing step of the occlusal plane surface (mentioned as “image producing step” hereinafter) 106 and the step of displaying the occlusal plane surface image so as to face each other (mentioned as “displaying step” hereinafter) 107 are sequentially executed.

The occlusal surface specifying step of the present embodiment comprises the step (100-103) in which the basic image which becomes a basis for the specifying the occlusal surface S is produced from the X-ray absorption coefficient distribution information V and displayed on the displaying portion 10, and the step (104, 105 in which the occlusal surface S is specified by the sifting operation of the horizontal section setting cursor C1 on the basic image.

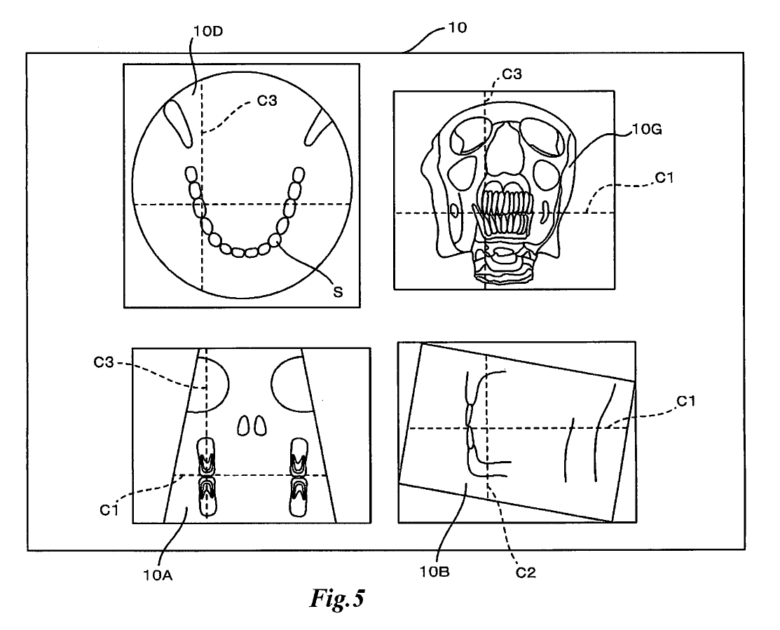

The basic image is at least one of the front sectional image 10A, the side sectional image 10B and the basic volume rendering image 10G (FIG. 5) mentioned hereinafter.

FIG. 3 shows the X-ray absorption coefficient distribution information V of the head of the object which is reconstructed and produced from the X-ray image data of the head by the backward projection. The X-ray absorption coefficient distribution information V is in a cylindrical shape, because the basic X-ray projection image of the CT reconstructing is in a rectangular shape. The X-ray absorption coefficient distribution information V is processed by an image-processing so as to be rotated by θ degrees around the X-axis to the back side of the head of the object in order to display the layer plane on the basis of the occlusal surface S as the horizontal sectional image 10C, 10D.

The front sectional image 10A and the side sectional image 10B, which are the basic images in the occlusal surface specifying step, are obtained by slicing the X-ray absorption coefficient distribution information V by the predetermined X layer plane and Y layer plane after the X-ray absorption coefficient distribution information V has been directed and described in the X-axis, Y-axis, Z-axis coordination. In this specifications, as an example, the embodiment of the present invention shall be explained with the body axis direction of the object as the Z-axis, the front and back direction of the object as the Y-axis, and the direction perpendicular to the Z-axis and the Y-axis as the X-axis.

The front sectional image 10A is obtained by slicing the X-ray absorption coefficient distribution information V by the Y layer plane. The side sectional image 10B is obtained by slicing the X-ray absorption coefficient distribution information V by the X layer plane.

The horizontal sectional image 10D is obtained by slicing the X-ray absorption coefficient distribution information V by the Z layer plane which is on the basis of the layer plane corresponding to the specified occlusal surface S in the occlusal surface specifying step.

The X-ray CT apparatus or the X-ray CT image displaying apparatus, or alternately, the operator, can recognize the facing direction of the object on the basis of the starting angle of the X-ray CT, because the X-ray CT is executed while the object is kept in the specified location and the specified direction. Therefore it is preferable to direct and describe the X-ray absorption coefficient distribution information V in the specified direction and the coordination, after rotating the X-ray absorption coefficient distribution information V so as to direct the portion such as dental arch of the object into the specified direction.

The horizontal sectional image 10C shown in FIG. 1 is not shown here, however it is not necessary to say that such a horizontal sectional image 10C is also obtained by slicing the X-ray absorption coefficient distribution information V by the Z layer plane which is on the basis of the layer plane corresponding to the specified occlusal surface S in the occlusal surface specifying step.

The above procedures are executed in the steps of the step 101 where the X-ray absorption coefficient distribution information is produced, the step 102 where the basic image is produced, and the step 103 where the basic image is displayed, respectively as shown in the flow chart of FIG. 2,

Then, the horizontal section setting cursor C1, the longitudinal section setting cursor C2, and the cross section setting cursor C3 are displayed on the front sectional image 10A and the side sectional image 10B (cursor displaying step 104 in the flow chart of FIG. 2).

The horizontal section setting cursor C1 is provided in order to set the Z layer plane of the X-ray absorption coefficient distribution information V so that the horizontal sectional image 10C, 10D are obtained by slicing the X-ray absorption coefficient distribution information V by the Z layer plane which is specified by the horizontal section setting cursor C1.

The longitudinal section setting cursor C2 is provided in order to set the Y layer plane of the X-ray absorption coefficient distribution information V so that the front sectional image 10A is obtained by slicing the X-ray absorption coefficient distribution information V by the Y layer plane which is specified by the longitudinal section setting cursor C2.

Further, the cross section setting cursor C3 is provided in order to set the X layer plane of the X-ray absorption coefficient distribution information V so that the side sectional image 10B is obtained by slicing the X-ray absorption coefficient distribution information V by the X layer plane which is specified by the cross section setting cursor C3.

As for the production of these slice image, the other several slice images produced and prepared in advance may be selectively displayed according to the operations of the cursor C1-C3, besides the slice images produced at each time of the real time processing according to the operations of the cursor C1-C3.

Above procedures are executed in the step 104 in the flow chart in FIG. 2, where the cursor is displayed.

Then, the occlusal surface S is specified by shifting the horizontal section setting cursor C1 on the front sectional image 10A or the side sectional image 10B (step 105 in the flow chart of FIG. 2).

The occlusal surface S may be specified by inputting the coordination value while the front sectional image 10A or the side sectional image 10B is watched. More particularly, the occlusal surface S is specified by inputting the Z-axis coordination value, because the occlusal surface S is constituted as a horizontal section.

If the horizontal section setting cursor C1 is shifted by the operation, then the horizontal section setting cursor C1 displayed on the front sectional image 10A and the horizontal section setting cursor C1 displayed on the side sectional image 10B are shifted in conjunction with each other.

When the occlusal surface S is specified on the front sectional image 10A or the side sectional image 10B, the upper and lower occlusal plane surface images on the basis of the occlusal surface S is produced (image producing step 106 in the flow chart of FIG. 2), and displayed so as to face each other (displaying step 107 in the flow chart of FIG. 2).

The volume rendering image 1E, 10F seen upwardly and downwardly from the inside of the oral cavity of the jaw on the basis of the occlusal surface S according to the X-ray absorption coefficient distribution information V, or the horizontal sectional image 10C, 10D obtained by slicing the X-ray absorption coefficient distribution information V by the Z layer plane on the basis of the occlusal surface S are shown as the upper and lower occlusal plane surface images (as shown in FIG. 1).

In the image producing step, the volume rendering image 10E, 10F are produced by rendering the upper side and the lower side of the specified occlusal surface S according to the X-ray absorption coefficient distribution information V which is obtained by the image-processing to incline the X-ray absorption coefficient distribution information V by −θ degrees around the X-axis clockwise against the watching direction of the FIG. 4 as shown in the figure.

FIG. 4 shows the X-ray absorption coefficient distribution information V sliced by the X layer plane in order to make it easy to explain the basic concept of the volume rendering.

Hereinafter, the method of producing the volume rendering image is explained as an example in which the images are produced from the occlusal surface S in the Z-axis direction after the rendering direction is specified to the Z-axis direction which is perpendicular to the occlusal surface S. However, as the embodiment of the present invention, the rendering direction is not necessarily limited to the Z-axis direction. For example the image may be produced and displayed on the displaying portion while the rendering direction is set for other direction than the Z-axis direction, after the X-ray absorption coefficient distribution information V is divided into upper side and lower side of the occlusal surface S on the basis of the specified occlusal surface S.

As shown in FIG. 4, the rendering of the upper side of the occlusal surface S is carried out in the manner that the plural of surface S1 (#1, #2, . . . , #n) with the specified interspaces are specified from the specified occlusal surface S in the direction of the arrow

A which is parallel to the Z-axis direction. The voxel value of the point P1(#1, #2, . . . , #n) on the surface S1 (#1, #2, . . . , #n) are sampled in the direction of the arrow A from the point P on the specified occlusal surface S. Then these voxel values are arithmetically added for all of the points on the occlusal surface S. Thus it becomes possible to produce the volume rendering image 10E which is upwardly seen from the inside of the oral cavity of the jaw on the basis of the occlusal surface S.

On the other hand, the rendering of the lower side of the occlusal surface S is carried out in the manner that the plural of surface S2 (#1, #2, . . . , #n) are specified from the specified occlusal surface S in the direction of the arrow B which is the opposite direction of the arrow A and parallel to the Z-axis direction with the specified interspaces. The voxel values of the point P2 (#1, #2, . . . , #n) on the surface S2 (#1, #2, . . . , #n) are sampled in the direction of the arrow B from the point P on the specified occlusal surface S. Then these voxel values are arithmetically added for all of the points on the occlusal surface S. Thus it becomes possible to produce the volume rendering image 10E which is downwardly seen from the inside of the oral cavity of the jaw on the basis of the occlusal surface S.

When the sampled voxel values are arithmetically added, the voxel value may be preferably multiplied by the coefficient which becomes smaller when the distance from the occlusal surface S becomes larger, so that the closer part to the occlusal surface S becomes clearer.

Further, if the produced upper and lower occlusal plane surface images (the horizontal sectional image 10C, 10D or the volume rendering image 10E, 10F) are displayed by color-coding according to the X-ray absorption coefficient, in the color-code displaying step, then the differences in the teeth, the dental pulp, the gingiva, the implant, and the filling can be easily recognized, so that they can be understood more intuitively and conveniently.

Thus the occlusal surface S of the object is specified by using the cursor on the side sectional image 10B which is produced from the X-ray absorption coefficient distribution information V, and the occlusal plane surface images upwardly and downwardly seen from the inside of the oral cavity of the jaw on the basis of the specified occlusal surface S are displayed so as to face each other.

Therefore, the operator can easily specify the occlusal surface S and understand the condition of the upper and lower jaw visibly. Further, both of the patient and the operator can understand the location of the teeth sensuously. In addition, the upper jaw is displayed as seen from the foot side of the patient and the lower jaw is displayed as seen from the head side of the patient. Therefore both of the operator and the patient can recognize the image as if they were observing the inside of the oral cavity of the patient who actually opened the mouth, so that both of the operator and the patient can understand the condition of the oral cavity or the location of the teeth more intuitively.

If the operator additionally specifies the occlusal surface S by shifting the horizontal section setting cursor C1 after the occlusal plane surface images upwardly and downwardly seen from the inside of the oral cavity of the jaw on the basis of the specified occlusal surface S are displayed so as to face each other through the occlusal surface specifying step, the image producing step, and the displaying step, then the detail portion of the jaw can be observed well and conveniently.

The number of such an additional specified occlusal surface S is not limited to one. It is possible to specify a plural of additional specified occlusal surfaces S. In that case, if the upper and lower occlusal plane surface images corresponding to the specified plural occlusal surfaces S are memorized sequentially in the recoding apparatus of the X-ray CT image displaying apparatus and so on, then the images can be selectively read out and displayed, or the past conditions of the occlusal plane surface can be conveniently recognized as an image after the time has passed.

FIG. 5 shows the other example of the occlusal surface specifying step to be explained. In this embodiment, in addition to the front sectional image 10A and the side sectional image 10B, the basic volume rendering image 10G obtained from the X-ray absorption coefficient distribution information V by volume rendering is displayed as the basic image for specifying the occlusal surface S.

Such a basic volume rendering image can be obtained by the volume rendering of the X-ray absorption coefficient distribution information V on the basis of the selected basic plane in the X-Y-Z coordination system.

In this embodiment, the basic volume rendering image 10G of the head of an object is displayed on the basis of the basic plane which is parallel to the Z-axis locating in front of the object.

On such a basic volume rendering image 10G, the occlusal surface S is specified by the shifting operation of the horizontal section setting cursor C1. Once the occlusal surface S is specified, then the upward and downward occlusal plane images seen upwardly and downwardly from inside of the oral cavity of the jaw of the head of an object are produced and displayed on the basis of the occlusal surface S in the image producing step (106 in FIG. 2).

As such upper and lower occlusal plane surface images, the horizontal sectional image 10C, 10D or the volume rendering image 10E, 10F are displayed. However as for the producing method of these images, the explanation is omitted here, because the method has been already mentioned above. Then, in the displaying step (107 in FIG. 2), the upper and lower occlusal plane surfaces images are displayed so as to face each other.

In FIG. 5, only the horizontal sectional image downwardly seen from the inside of the oral cavity of the jaw 10D is displayed as the occlusal plane surface image. However it is possible to display the upper and lower occlusal plane surface images so as to face each other, for example by the operation of a certain switch which is named such as “up and down displaying”.

FIG. 6 shows the other example of the embodiment, in which the occlusal plane surface images are displayed so as to face each other. In this embodiment, the volume rendering image 10H of the whole head of the object including the upper and lower occlusal plane surface images which show the temporomandibular joint opening by less than 180 degrees, is displayed so as to be able to rotate around an arbitrary point in the X-Y-Z coordination system in the displaying step as shown in FIG. 6 (a)-(d).

Such a volume rendering image 10H can be constituted by the image-processing of the volume rendering image 10E, 10F produced on the basis of the occlusal surface S. In this volume rendering image 10H of the whole head of the object, the right and left rotation keys (not shown in the figure) are displayed as overlapped in the image, and the image can rotate around the arbitrary point in the X-Y-Z coordination system in any desired directions of the coordination system.

Thus the volume rendering image 10H of the whole head of the object includes the volume rendering images upwardly and downwardly seen from the inside of the oral cavity of the jaw 10E, 10F which display the temporomandibular joint so as to open by less than 180 degrees, and the rotation keys (not shown in the figure) are displayed in order to make the image rotate around a arbitrary point in the X-Y-Z coordination system in the desired directions of the coordination system.

Therefore, both of the operator and the patient can recognize the image as if they were observing the inside of the oral cavity of the patient who actually opened the mouth, so that both of the operator and the patient can understand the condition of the oral cavity or the location of the teeth more precisely and intuitively.

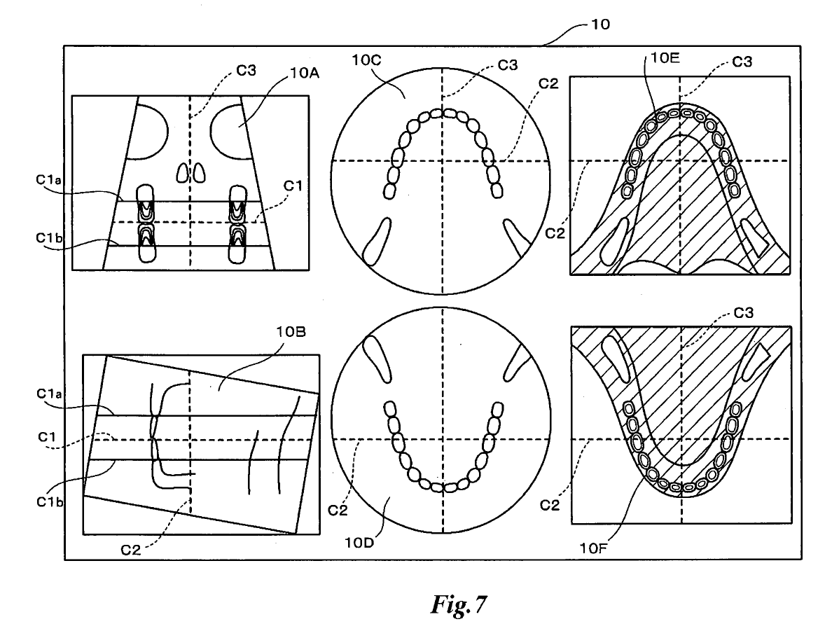

FIG. 7 shows the other example of the embodiment displaying the basic image and the occlusal plane surface images upwardly and downwardly seen from the inside of the oral cavity of the jaw in the displaying portion.

This embodiment is same as that shown in FIG. 1 at the following points. Namely, this embodiment is same as that at the point that the front sectional image 10A and the side sectional image 10B are displayed as the basic image, at the point that the horizontal sectional image 10C, 10D and the volume rendering image 10E, 10F are displayed as the occlusal plane surface image, and at the point that each of the longitudinal section and the cross section of the X-ray absorption coefficient distribution information V are specified by the shifting operation of the longitudinal section setting cursor C2 and the cross section setting cursor C3.

However the embodiment of FIG. 7 is different from the embodiment of FIG. 1 at the following points. One point is that the mutually different first occlusal surface S and the second occlusal surface S are specified on the front sectional image 10A and the side sectional image 10B in the occlusal surface specifying step by displaying the first horizontal section setting cursor C1a which shifts upwardly and downwardly and the second horizontal section setting cursor C1b which shifts upwardly and downwardly. Another point is that the occlusal plane surface images on the basis of the first and the second occlusal surface S are produced as the occlusal plane surface images in the image producing step.

The first and the second horizontal section setting cursor C1a, C1b are independently shifted both upwardly and downwardly. Once the different occlusal surface S1, S2 are specified by the shifting operation of the cursors (the occlusal surface specifying step), then in the step 106 (the image producing step) of FIG. 2, the horizontal sectional image upwardly seen from the inside of the oral cavity of the jaw 10C on the basis of the occlusal surface S specified by the first horizontal section setting cursor C1a is produced and also the horizontal sectional image downwardly seen from the inside of the oral cavity of the jaw 10D on the basis of the occlusal surface S specified by the second horizontal section setting cursor C1b are produced.

Alternatively, in the image producing step, the volume rendering image upwardly seen from the inside of the oral cavity of the jaw 10E is produced by the upward volume rendering in the Z-axis direction on the basis of the occlusal surface S specified by the first horizontal section setting cursor C1a, and the volume rendering image downwardly seen from the inside of the oral cavity of the jaw 10F is produced by the downward volume rendering in the Z-axis direction on the basis of the occlusal surface S specified by the second horizontal section setting cursor C1b.

Then, in the displaying step, the horizontal sectional image 10C, 10D and the volume rendering image 10E, 10F are displayed so as to face each other. In this displaying step, the horizontal sectional image 10C, 10D and the volume rendering image 10E, 10F are displayed as the occlusal plane surface image, however the present invention is not limited to this example of the embodiment. Either the horizontal sectional image or the volume rendering image may be displayed as the occlusal plane surface image.

Also in this example, in the displaying step, the volume rendering image 10H of the head of the object including the volume rendering image 10E, 10F may be displayed so as to rotate in any direction in the X-Y-Z coordination (FIG. 6) after being produced by the image-processing.

Further, the occlusal surface S may be specified by the shifting operation of the first and the second horizontal section setting cursors C1a, C1b after the first and the second horizontal section setting cursor C1a, C1b are displayed as the basic images on the basic volume rendering image 10G (FIG. 5).

Further, the specification by the horizontal section setting cursor C1a, C1b may be adapted only to a part of area of the occlusal surface images. Namely, a part of area is specified by circle or rectangle (not shown) on the occlusal surface images, and the setting operation of the first and second horizontal section setting cursors C1a, C1b can affect only to such specified area. In this case, the occlusal surface images specified by the first and second horizontal section setting cursors C1a, C1b are displayed as FIG. 7 as for the specified part of area by circle or rectangle, and the occlusal surface images remain according to the original specified occlusal surface S as FIG. 1 as for the other part of such specified part of area, therefore, two kinds of images are displayed with superimposing. According to this structure, for example, the basic occlusal surface images are remained, and the specific teeth or area can be observed with moving the position of occlusal surface from tooth crown to the root.

These occlusal plane surface images are not limited to the examples shown in FIG. 1. These occlusal plane surface images are on the different basis of the different occlusal surfaces S specified by the independent shifting operations of the first and the second horizontal section setting cursors C1a, C1b. However, if the first and the second horizontal section setting cursors C1a, C1b are overlapped, then the occlusal plane surface images on the basis of the specified occlusal surfaces S are produced and displayed so as to face each other after the horizontal section setting cursor C1 is constituted with the first and the second horizontal section setting cursors C1a, C1b.

In this embodiment, the first and the second horizontal section setting cursors C1a, C1b are independently operated to shift, however they may be operated in conjunction with each other, for example in such a manner that the first horizontal section setting cursor C1a is operated to shift upwardly for a certain distance, then the second horizontal section setting cursor C1b is shifted downwardly for the same distance at the same time. Further, each different occlusal surfaces S may be specified by inputting the coordination parameters. Or the occlusal surfaces S may be alternatively specified by inputting the upper and lower distances (mm for example) of the first and the second horizontal section setting cursors C1a, C1b from the occlusal surface S in case of the embodiment in which the occlusal surface S is automatically specified as mentioned hereinafter.

Thus each occlusal plane surface image on the basis of each occlusal surface S can be displayed after specifying the different two occlusal surface S by shifting operation of the first and the second horizontal section setting cursors C1a, C1b, so that the convenient and more precise diagnosis becomes possible when the operator want to observe the occlusal plane surface image of the root portion of the tooth in the upper and lower jaw.

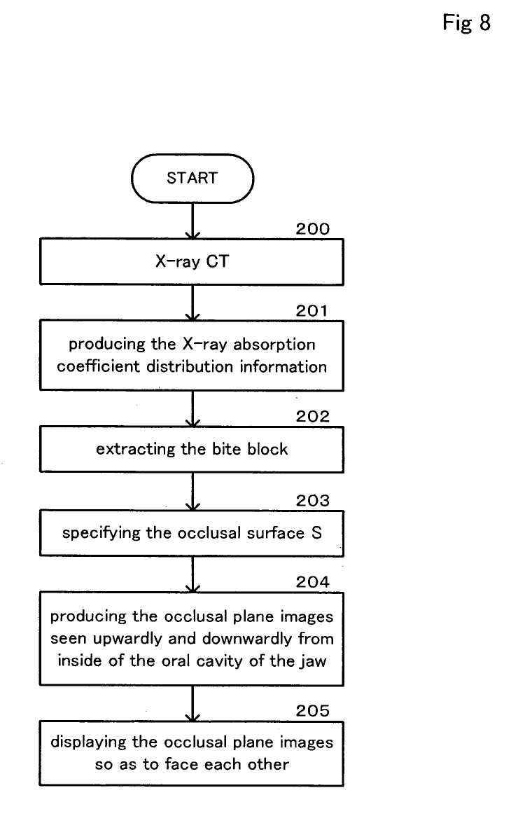

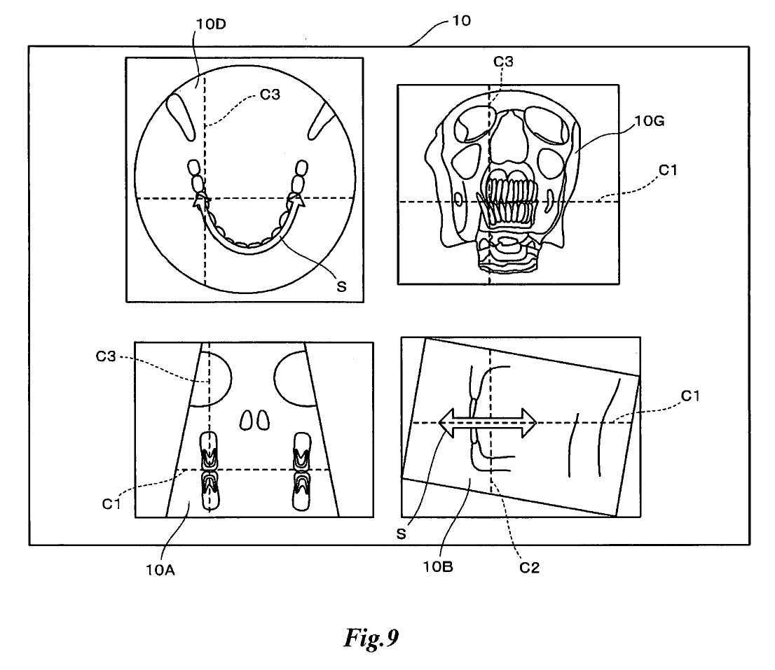

FIG. 8 shows the flow chart to explain the other example of the X-ray CT image displaying method of the present invention (200-205). FIG. 9 shows the other example of the displaying embodiment of the basic image and the occlusal plane surface image upwardly and downwardly seen from the inside of the oral cavity of the jaw on the displaying portion. In FIG. 9, only the downward occlusal plane image seen downwardly from the inside of the oral cavity is displayed and the upward occlusal plane surface image is not displayed. Here in FIG. 9, the upward occlusal plane surface image is not shown, because both of the upward and downward occlusal plane surface images can be displayed in any way just as FIG. 1 and FIG. 7.

According to this other example of the X-ray CT image displaying method, the X-ray absorption coefficient distribution information V of the head of the object is produced by backward projection of the X-ray image data of the head of the object obtained by the X-ray CT in which the objective patient bites the bite block in advance. Then the occlusal surface specifying step is executed. In the occlusal surface specifying step (200-202), the bite block is extracted by the image-analysis of the produced X-ray absorption coefficient distribution information V.

The bite block is constituted so as to be bitten by the objective patient. In order to extract the bite block by the image-analysis, the bite block comprises a board with approximately 5 mm thickness containing 3 to 5 pieces of ceramic ball of which X-ray absorption coefficient is already known. The occlusal surface S is judged from the location of the ceramic ball. Not only ceramic ball but also any material of which X-ray absorption coefficient is already known in advance, can be adopted for the bite block.

Then, the occlusal surface S is judged from the extracted bite block and the layer plane corresponding to the specified occlusal surface S is figured out from the X-ray absorption coefficient distribution information V.

The occlusal surface specifying step is carried out just as mentioned above and then the image producing step and the displaying step are carried out, so that the occlusal plane surface image upwardly and downwardly seen from the inside of the oral cavity of the jaw on the basis of the layer plane figured out are produced and displayed so as to face each other (203-205).

FIG. 9 shows the front sectional image 10A, side sectional image 10B, and the basic volume rendering image 10G as the basic images, each of which displays the horizontal section setting cursor C1, the longitudinal section setting cursor C2, and the cross section setting cursor C3 respectively. As for the function of the horizontal section setting cursor C1, the function of the longitudinal section setting cursor C2, the function of the cross section setting cursor C3, the switching display by the shifting operation of these cursors, and the displaying method of the occlusal plane surface image, the explanations are omitted because they are similar to the embodiment shown in FIG. 1 and FIG. 7. In this embodiment, the basic image is not necessarily displayed because the occlusal surface S is automatically specified.

In this embodiment, the occlusal surface S is schematically displayed using the white arrow on the side sectional image 10B and the horizontal sectional image 10D where the occlusal surface S is specified by the bite block extracted from the X-ray absorption coefficient distribution information V by image-analysis, so that the operator can visibly understand the occlusal surface S. As for the image of the bite block, it may be deleted by the image-processing.

Thus according to this embodiment, the bite block is extracted from the X-ray absorption coefficient distribution information V of the head of the object by image-analysis, and the occlusal plane surface image on the basis of the layer plane corresponding to the specified occlusal surface S is produced and displayed from the extracted bite block so as to face each other. Therefore, it is convenient for the operator because he does not have to specify the occlusal surface S.

When the horizontal section setting cursor C1 is displayed on the side sectional image 10B by combining the present invention to the X-ray CT image displaying method in which the occlusal surface S is specified by sifting operation of the horizontal section setting cursor C1 on the front sectional image 10A or the side sectional image 10B, then the operator can visibly understand the approximate location of the occlusal surface S, if the horizontal section setting cursor C1 is displayed on the occlusal surface S which is specified by the bite block.

Then the detail portion of the jaw can be observed while the occlusal surface S is corrected by the small adjustment of the horizontal section setting cursor C1, so that the condition of the root portion of the tooth, the dental pulp, the implant, the gingiva, the teeth, and so on, can be understood more intuitively and conveniently.

Further, the method to produce and display the different upper and lower occlusal plane surface images by the first and the second horizontal section setting cursors which have been explained in the embodiment mentioned above can be used together with, if the other horizontal section is specified besides the occlusal surface S which is specified by the bite block.

In addition, according to the present embodiment, the occlusal surface S can be specified automatically by using the bite block, so that the plane upwardly shifted (mm) and downwardly shifted (mm) can be further automatically specified on the basis of the specified occlusal surface S as the first and the second occlusal surface S. Therefore, the occlusal plane surface image can be automatically produced and displayed on the basis of the first and the second occlusal surface S.

In this case, the occlusal surface S and the first and the second occlusal surface S can be specified automatically, and the occlusal plane surface image can be automatically produced and displayed, so that the operator or the patient can conveniently save the troubles of the operation.

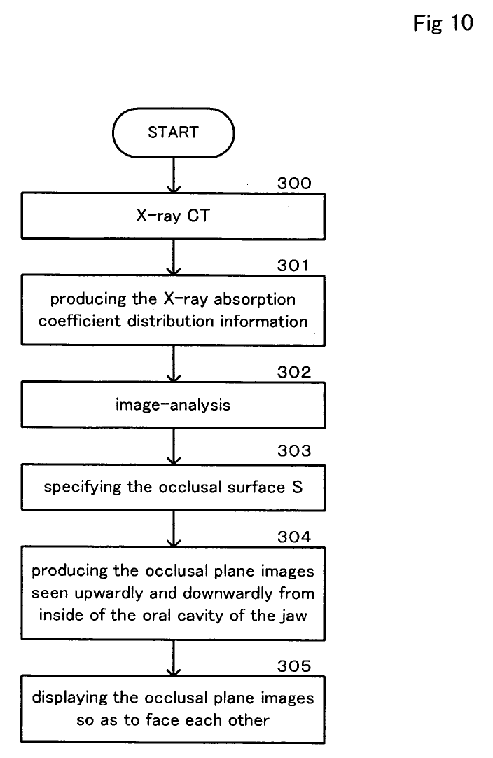

FIG. 10 shows the flow chart (300-305) to explain the other example of the X-ray CT image displaying method of the present invention, in which the occlusal surface S is automatically specified.

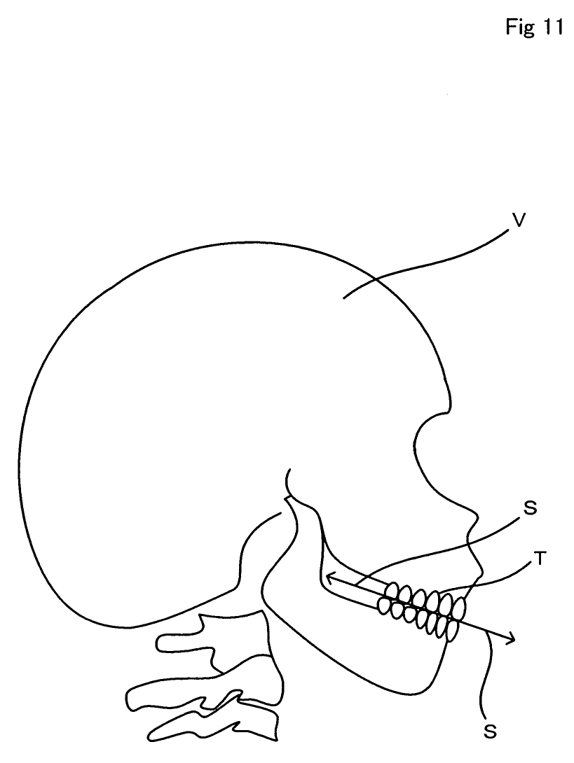

FIG. 11 shows the schematic drawing of the X-ray absorption coefficient distribution information V of the side head of the object to explain the automatic specifying of the occlusal surface S.

In this X-ray CT image displaying method, the X-ray absorption coefficient distribution information V is produced by the back projection of the X-ray image data of the head of the object, and the occlusal surface specifying step is carried out.

In the occlusal surface specifying step, the occlusal surface S is specified by the image-analysis of the produced X-ray absorption coefficient distribution information V (300-303).

The example of the image-analysis to specify the occlusal surface S is as follows. The outline of the internal construction of the object which includes the outline of the side section of the dental arch of the object T is extracted by the edge-extracting for the X-ray absorption coefficient distribution information V of the side head of the object shown in FIG. 11. Then the occlusal surface S is specified as the horizontal section where the dental arch T locates, after the dental arch T is specified by specifying the outline of the side section of the dental arch T, or alternatively by tracing the air zone existing between the upper and lower dental arch T.

When the dental arch T is specified by the outline, for example, it is possible to distinguish the hard tissue of the head, the soft tissue of the head, and the air zone by binarizing the X-ray absorption coefficient distribution information V.

In this case of the binarization, the side sectional image of the head of the object is displayed by color-coding clearly. For example the hard tissue is displayed with white color and the soft tissue and the air zone is displayed with black color.

Then, the layer plane corresponding to the specified occlusal surface S is figured out from the X-ray absorption coefficient distribution information V. The occlusal surface specifying step 300-303) is just as mentioned above. Then, after the image producing step (304) and the displaying step (305) is executed, the occlusal plane surface images upwardly and downwardly seen from the inside of the oral cavity of the jaw 10E, 10F are produced and displayed so as to face each other on the basis of the layer plane figured out.

Thus according to this embodiment, the occlusal surface S is specified from the X-ray absorption coefficient distribution information V by image-analysis, and the occlusal plane surface image on the basis of the layer plane corresponding to the specified occlusal surface S is produced and displayed so as to face each other, so that it is convenient for the operator because he does not have to specify the occlusal surface S.

The X-ray absorption coefficient distribution information V shown in FIG. 11 may not necessarily be displayed because it is shown as an example in order to explain the invention understandably.

When the present embodiment is combined in the X-ray CT image displaying method in which the occlusal surface S is specified by shifting operation of the horizontal section setting cursor C1 on the front sectional image 10A or the side sectional image 10B on which the horizontal section setting cursor C1 is displayed, the operator can visibly understand the approximate location of the occlusal surface S without using the bite block, if the horizontal section setting cursor C1 is displayed on the occlusal surface S specified by image-analysis. Then afterwards, the detail of the jaw portion can be observed while the occlusal surface S is corrected by the slight adjustment of the horizontal section setting cursor C1, so that the conditions of the root portion of the tooth, the dental pulp, the implant, the gingiva and the teeth can be understood more intuitively and conveniently.

Once after the occlusal surface S is specified, the detail image of the jaw can be observed upwardly and downwardly by specifying the further additional occlusal surface S. Alternatively, the additional occlusal surface S may be specified by the operator at will, or may be automatically specified according to the predetermined set value.

These points are the same as the embodiment mentioned above in which the occlusal surface S is specifies by the bite block.

Hereinafter, the examples of the X-ray CT apparatus according to the present invention is explained as a apparatus to carry out the displaying method mentioned above.

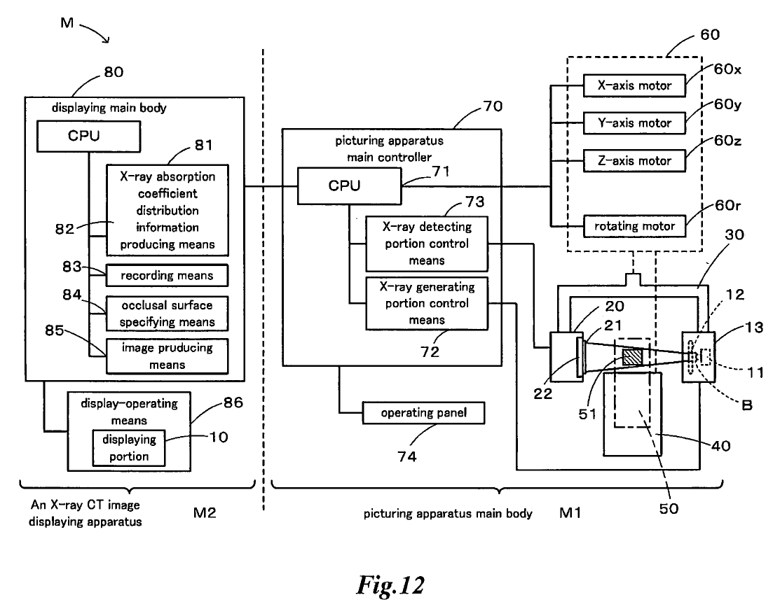

FIG. 12 shows the block diagram to explain the basic construction of the X-ray CT apparatus M including the X-ray CT image displaying apparatus of the present invention. The X-ray CT apparatus M comprises the X-ray picturing apparatus main body M1 and the X-ray CT image displaying apparatus M2. In FIG. 12, the X-ray picturing apparatus main body M1 and the X-ray CT image displaying apparatus M2 is constituted to transmit and receive the data through the communication cable. However the data can be transmitted or received wirelessly or via recording medium. Or alternatively, the X-ray CT image displaying apparatus M2 may be provided in the X-ray picturing apparatus main body M1. These embodiments can be designed as necessary considering the location or the environment of the apparatus.

The X-ray picturing apparatus main body M1 comprises the picturing means 30 fixing the X-ray generating portion 13 and the X-ray detecting portion 20 so as to face each other, the object holding means 40 holding the patient as the object 50, the driving portion 60 driving the picturing means 30 and the object holding means 40, and the picturing apparatus main controller 70 having the operating panel 74.

The X-ray generating portion 13 comprises the X-ray generator 11 such as an X-ray tube irradiating X-ray and the irradiation field control means 12 such as slit limiting broadness of the conical X-ray beams B. The X-ray detecting portion 20 comprises the cassette 22 having the X-ray detector 21 such as two dimensional CCD sensor. The driving portion 60 comprises the X-axis motor 60 x and the Y-axis motor 60 y both shifting the rotation axis of the picturing means 30 horizontally, the Z-axis motor 60z shifting the picturing means 30 and the object holding means 40 upwardly and downwardly, and the rotating motor 60r rotating the picturing means 30. Alternatively, the X-axis motor 60x and the Y-axis motor 60y may be designed to shift the object holding means 40 horizontally.

The picturing apparatus main controller 70 comprises the CPU 71 executing the plural kinds of control program which includes the control program to controls the driving portion 60, the X-ray generating portion control means 72 controlling the X-ray generating portion 13, and the X-ray detecting portion control means 73 controlling the X-ray detecting portion 20.

The operating panel 74 comprises the CPU 71 and the X-ray generating portion control means 72, or a small liquid crystal panel or a plural of keys to specify the conditions for the X-ray detecting portion control means 73 to work.

The X-ray CT main unit M1 takes the X-ray projection image of the head of the object according to the commands from the operating panel 74 or the displaying apparatus M2, while setting the photographed area 51 of the object 50 between the X-ray generating portion 13 and the X-ray detecting portion 20, and rotating for example by 180 degrees.

The X-ray picturing apparatus main body M1 receives the commands and the coordinate data and so on from the operating panel 74 or the displaying apparatus M2, while it transmits the acquired projection image to the displaying apparatus M2.

On the other hand, the displaying apparatus M2 consists of for example a work station comprising the displaying main body 80 with the liquid crystal display monitor, the key board, the mouse and so on, and the display operation means 86 including the displaying portion 10.

The displaying main body 80 comprises the CPU 81 executing the plural kinds of the programs, the X-ray absorption coefficient distribution information producing means 82 reconstructing the X-ray projection image which is received from the picturing apparatus main body M1 by backward projection so as to produce the X-ray absorption coefficient distribution information V, the recording means 83 such as a hard disk memorizing the photographed data or the image data and so on, the occlusal surface specifying means 84, and the image producing means 85.

The occlusal surface specifying means 84 comprises the means which produces and displays at least one of the front sectional image 10A, the side sectional image 10B and the basic volume rendering image 10G as the basic image on the displaying portion 10 from the X-ray absorption coefficient distribution information V of the head of the object. The occlusal surface specifying means 84 also comprises the means which further displays the horizontal section setting cursor C1 (the horizontal section setting cursor C1 includes the first and the second horizontal section setting cursors C1a, C1b as hereinafter), the longitudinal section setting cursor C2 and the cross section setting cursor C3, and the occlusal surface specifying means 84 which specifies the occlusal surface S at least by the shifting operation of the horizontal section setting cursor C1.

The occlusal surface specifying means 84 specifies the different occlusal surfaces S by shifting the first horizontal section setting cursor C1a and the second horizontal section setting cursor C1b independently on at least one of the images of the front sectional image 10A, the side sectional image 10B, and the basic volume rendering image 10G.

The image producing means 85 produces the occlusal plane surface image upwardly and downwardly seen from the inside of the oral cavity of the jaw on the basis of the specified occlusal surface S according to the X-ray absorption coefficient distribution information V of the object, when the occlusal surface S is specified by the shifting operation of the horizontal section setting cursor C1 on the one of the front sectional image 10A, the side sectional image 10B, and the basic volume rendering image 10G.

When the different first and the second occlusal surface S are specified by the shifting operation of the first and the second horizontal section setting cursors C1a, C1b respectively, the occlusal plane surface image upwardly seen from the inside of the oral cavity of the jaw is produced on the basis of the occlusal surface S specified by the first horizontal section setting cursor C1a. Also the occlusal plane surface image downwardly seen from the inside of the oral cavity of the jaw is produced on the basis of the occlusal surface S specified by the second horizontal section setting cursor C1b.

The display operation means 86 comprising the displaying portion 10 displays the upper and lower occlusal plane surface images produced by the image producing means 85 so as to face each other.

The display operation means 86 can be convenient to recognize the condition of the teeth, the dental pulp, the gingiva, the implant, the filling and so on, if it is provided with the function to display the images by color-coding, according to the X-ray absorption coefficient, after the occlusal plane surface image (claim 12 is produced.

The above mentioned X-ray CT image displaying apparatus M2 produces and displays at least one of the front sectional image 10A, the side sectional image 10B and the basic volume rendering image 10G as the basic image from the X-ray absorption coefficient distribution information V produced by reconstructing the X-ray image data obtained by the X-ray CT of the head. The X-ray CT image displaying apparatus M2 also produces and displays the upper and lower occlusal plane surface images so as to face each other on the basis of the occlusal surface S specified by the shifting operation of the horizontal section setting cursor C1 on each images (claim 7-9). However the X-ray CT image displaying apparatus M2 is not limited to the constitution above. The X-ray CT image displaying apparatus M2 may be constituted as follows.

The occlusal surface specifying means 84 may figure out the layer plane corresponding to the specified occlusal surface S which is specified by extracting the bite block from the X-ray absorption coefficient distribution information V by the image-analysis (claim 10).

Further, the occlusal surface specifying means 84 may figure out the layer plane corresponding to the specified occlusal surface S which is specified from the X-ray absorption coefficient distribution information V by the image-analysis (claim 11).

According to these constitutions, the occlusal surface S can be specified automatically without the cursor operation by the operator, so that the above mentioned basic image (at least one of the front sectional image 10A, the side sectional image 10B, and the basic volume rendering image 10G) and the horizontal section setting cursor C1 are not necessarily displayed on the displaying portion 10. However it is not necessary to say that these basic image or the horizontal section setting cursor may be displayed before fact or after fact, so that the operator or the patient can slightly adjust the shift of the occlusal surface S which is once automatically specified.

The X-ray CT apparatus M1 having the X-ray CT image displaying apparatus M2 (claim 13) is explained as above, however, the effect of the present invention can be also achieved only by the X-ray CT image displaying apparatus M2 (claim 7-12), if the X-ray image data of the object is once acquired from the X-ray CT.

1. An X-ray CT image displaying method for displaying X-ray CT image of the head of an object to be examined by X ray CT, comprising:
an occlusal surface specifying step of receiving an occlusal surface specifying in the X-ray image data obtained by X-ray CT of said head of an object to be examined;
an image producing step of producing from said X-ray image data an upward occlusal plane image seen upwardly from inside of the oral cavity of the jaw of said head of an object to be examined and a downward occlusal plane image seen downwardly from the inside of said oral cavity both on the basis of said occlusal surface specified by said occlusal surface specifying step; and
a displaying step of displaying said two upward and downward occlusal plane images produced by said image producing step in a manner that said upward and downward occlusal plane images face each other.

2. The X-ray CT image displaying method as set forth in claim 1, wherein:
in said occlusal surface specifying step, at least the surface specifying of a first occlusal surface and a second occlusal surface both of which are different from each other are received; and
in said image producing step, said upward occlusal plane image and said downward occlusal plane image are produced from said X-ray image data on the basis of at least said first occlusal surface and said second occlusal surface.

3. The X-ray CT image displaying method as set forth in claim 1 or 2, wherein:
said occlusal surface specifying step comprises:
a step of producing and displaying at least one of the front sectional image, the side sectional image, and the basic volume rendering image as a basic image from said X-ray absorption coefficient distribution information obtained from said X-ray CT of said head; and
a step of receiving an operation of a cursor to specify said occlusal surface on at least one of said front sectional image, said side sectional image, and said basic volume rendering image.

4. The X-ray CT image displaying method as set forth in claim 1 or 2, wherein:
said X-ray image data is an X-ray image data obtained by picturing said object which bites a bite-block;
wherein said X-ray CT image displaying method further comprises a step of reconstructing said X-ray image data for producing an X-ray absorption coefficient distribution information of said head of an object to be examined as a preprocessing for receiving said occlusal surface specifying; and wherein
in said occlusal surface specifying step, said bite-block is extracted from said X-ray absorption coefficient distribution information by image-analysis to specify said occlusal surface, and figures out the layer image corresponding to the specified occlusal surface from the X-ray absorption coefficient distribution information thus obtained.

5. The X-ray CT image displaying method as set forth in claim 1 or 2, wherein:
said X-ray CT image displaying method further comprises a step of reconstructing an X-ray projection image of the head to produce an X-ray absorption coefficient distribution information as a preprocessing for receiving said occlusal surface specifying; and
in said occlusal surface specifying step, said occlusal surface is specified by image-analysis, and the layer image corresponding to the specified occlusal surface thus specified is figured out from said X-ray absorption coefficient distribution information.

6. The X-ray CT image displaying method as set forth in claim 1 or 2, wherein:
said X-ray CT image displaying method further comprises the color-code displaying step of displaying said upward and downward occlusal plane images by the color-coding according to said X-ray absorption coefficient after producing said upward and downward occlusal plane images.

7. An X-ray CT image displaying apparatus for displaying X-ray CT image of the head of an object to be examined by X ray CT comprising:
an occlusal surface specifying means for specifying an occlusal surface in the X-ray image data obtained by X-ray CT for said head;
an image producing means for producing from said X-ray image data an upward occlusal plane image seen upwardly from inside of the oral cavity of said jaw and a downward occlusal plane image seen downwardly from the inside of said oral cavity both on the basis of said occlusal surface specified by said occlusal surface specifying means, and
a displaying means for displaying said upward and downward occlusal plane images produced by said image producing means in a manner that said upward and downward occlusal plane image face each other.

8. The X-ray CT image displaying apparatus as set forth in claim 7, wherein:
said occlusal surface specifying means specifies at least a first occlusal surface and a second occlusal surface, both of which are different from each other; and
said image producing means produces said upward occlusal plane image and said downward occlusal plane image from said X-ray image data on the basis of at least said first occlusal surface and said second occlusal surface.

9. The X-ray CT image displaying apparatus as set forth in claim 7 or 8, wherein:
said occlusal surface specifying means displays at least one of the front sectional image, the side sectional image, and the basic volume rendering image of said head as a basic image to specify said occlusal surface, each of said images being produced from the X-ray absorption coefficient distribution information produced by said X-ray CT of said head, and specifies said occlusal surface by said operation of a cursor on at least one of said front sectional image, said side sectional image, and said basic volume rendering image.

10. The X-ray CT image displaying apparatus as set forth in claim 7 or 8, wherein:
said X-ray image data is an X-ray image data obtained by picturing said object which bites a bite-block;
said X-ray CT image displaying apparatus further comprises an X-ray absorption coefficient distribution information producing means for reconstructing said X-ray image data to produce an X-ray absorption coefficient distribution information of said head, and
said occlusal surface specifying means extracts said bite-block from said X-ray absorption coefficient distribution information by image-analysis to specify said occlusal surface, and figures out the layer image corresponding to the specified occlusal surface from the X-ray absorption coefficient distribution information thus obtained.

11. The X-ray CT image displaying apparatus as set forth in claim 7 or 8, wherein:
said X-ray CT image displaying apparatus further comprises an X-ray absorption coefficient distribution information producing means for reconstructing an X-ray projection image of said head to produce an X-ray absorption coefficient distribution information; and
said occlusal surface specifying means specifies said occlusal surface by image-analysis to specify said occlusal surface, and figures out the layer image corresponding to the specified occlusal surface from said X-ray absorption coefficient distribution information.

12. The X-ray CT image displaying apparatus as set forth in claim 7 or 8, wherein:
said displaying means displays said upward and downward occlusal plane images by the color-coding according to said X-ray absorption coefficient after producing said upward and downward occlusal plane images, by said image producing means.

13. An X-ray CT apparatus comprises:
an X-ray CT main unit at least having an X-ray detecting portion and an X-ray producing portion which produces an X-ray cone beam; and
said X-ray CT image displaying apparatus as set forth in claim 7 or 8;
wherein the X-ray CT main unit sets the projection area of an object to be examined between said X-ray producing portion and said X-ray detecting portion, and rotates both of said X-ray producing portion and said X-ray detecting portion around said projection area for obtaining the X-ray CT image of a head of said object.
